(19) United States
(12) Patent Application Publication
(10) Pub. No.: US 2002/0119571 A1
(43) Pub. Date: Aug. 29, 2002

(54) Genetically modified T-cells, method for producing them and use thereof

(76) Inventors: Thomas Ritter, Berlin (DE); Hans-Dieter Volk, Berlin (DE); Markus Hammer, Koengernheim (DE); Christine Brandt, Berlin (DE); Grit Schroeder, Rostock (DE); Manfred Lehmann, Rostock (DE); Alexander Fluegel, Muenchen (DE)

Correspondence Address: OBLON SPIVAK MCCLELLAND MAIER & NEUSTADT PC, FOURTH FLOOR, 1755 JEFFERSON DAVIS HIGHWAY, ARLINGTON, VA, 22202, US

(21) Appl. No.: 10/068,916

(22) Filed: Feb. 11, 2002

Related U.S. Application Data
(63) Continuation of application No. PCT/DE01/02184, filed on Jun. 8, 2001.

(30) Foreign Application Priority Data
Jun. 9, 2000 (DE) DE 100 28 833.2

(51) Int. Cl. A61K 48/00; C12N 5/08; C12N 15/867

(52) U.S. Cl. 435/456; 435/372; 424/93.21

(57) ABSTRACT

T cells of a graft recipient are stimulated in-vitro by cells of a graft donor or by cells which express dominant MHC-molecules, while they are transduced with immuno-modulatory genes using gene transfer. Following the gene transfer the transduced T cells start to express immuno-modulatory genes. The gene transfer can be accomplished using retroviruses, other viral vector systems or liposomes. Due to the chosen set-up of the experiment, which leads to the generation and expansion of allospecific T cells, the T cells migrate, after the in-vivo application, specifically into the allogeneic graft as well as into the draining lymph nodes and are then able to express the immuno-modulatory genes there. Rejection of allogeneic grafts (cells, tissues, organs) can be successfully prevented, tolerance towards allogeneic grafts can be induced and maintained.

### BACKGROUND OF THE INVENTION

1. Field of the Invention

The present invention relates to in-vitro gene-modified T cells for the prevention of allogeneic transplant rejection in-vivo, a process for their production and their use.

2. Discussion of the Background

Despite the success with conventional immunosuppression using Cyclosporin A, FK506, glucocorticoids or OKT3 (monoclonal antibody (mab) against CD3), the problem of transplant rejection is not yet satisfactorily solved. A lifelong drug-induced immunosuppression almost always leads to serious side effects and it is only occasionally possible to prevent a chronic rejection completely. It is therefore an aim of the transplantation research to achieve a lifelong acceptance of a foreign organ with a short-term therapy. In animal models there are already some approaches, which come close to this demand. The detailed analysis of the rejected or tolerated tissue is the key to understanding these approaches. During acute rejection, it always shows a massive infiltration of the tissue with granulocytes, monocytes und lymphocytes. The fact that the depletion of CD3-positive cells by OKT3 protects the graft from rejection, shows the critical role of the T lymphocytes, thymus-dependent or originating lymphocytes) (Ode-Hakim, S.; DöZcke, W. D.; Kern, F.; Volk, H. D.; Reinke, P.; DTH-like mechanisms in late acute rejection-role of CD 4⁺ T lymphocytes. Transplantation 1996, 61:1233-1340). Immunodeficient SCID mice (without B and T cells) are also not able to reject an allogeneic organ. Within the T-cell population (T-lymphocytes originating in the thymus) T-helper cells seem to be the initiators of rejection. This could be shown in CD4 and CD8 T cell-depleted mice, respectively. CD4 and CD8 refer to specific surface markers on T helper cells. While the CD8-depleted mice reject the graft, the CD4-depleted cannot (Campos, L.; Naji, A.; Deli, B. C.; Kern, J. H.; Kim, J. I.; Barker, C. F.; Markmann J. F.; Survival of MHC-Deficient Mouse Heterotropic Cardiac Allografts. Transplantation 1995, 59:187-191). If we take the Th1 /Th2 paradigm (Mosmann, T. R. & Coffmann, R. L.; Th1 and Th2 cells: Different patterns of lymphokine secretion lead to different functional properties. Annual Reviews Immunology 1989, 7:145-173) as the basis, it is mostly Th1-cells that are involved in the early phase of rejection. Th1-cells are T cells with the phenotype T helper 1, Th2-cells are T cells with the phenotype T helper 2. In rat models an increase of characteristic Th1-cytokines (interferon IFN-γ, interleukin IL-2) could be shown with the help of semiquantitative polymerase chain reaction (PCR) (Siegling, A.; Lehmann, M.; Riedel, H.; Platzer, C.; Brock, J.; Emmrich, F.; Volk, H.-D.; A nondepleting anti-rat monoclonal antibody which suppresses T helper 1-like but not T helper 2-like intragraft lymphokine secretion induces long-term survival of renal allografts. Transplantation 1994a, 57: 464-467). CD4⁺ cells isolated from the graft produced, after in-vitro re-stimulation, also mainly Th1 cytokines.

All therapy protocols have the common aim to inhibit the potentially damaging Th1 cells in their origin and function. While the conventional methods try to achieve this aim with a global depletion or inhibition of the lymphocytes, more recent approaches intervene in the process of the T cell activation. Monoclonal antibodies against the CD4-receptor modify the signals via the T-cell receptor (TCR) signal (Lehmann, M.; Sternkopf, F.; Metz, F.; Brock, J.; Döcke, W.-D.; Plantikow, A.; Kuttler, B.; Hahn, H. J.; Ringel, B.; Volk, H.-D; A novel high-efficient anti-CD4 monoclonal antibody induces long-term survival of rat skin allografts. Transplantation 1992, 54: 959-962; 1992; Siegling, A., Lehmann, M.; Platzer, C.; Emmrich, F.; Volk, H.-D.; A novel multispecific competitor fragment for quantitative PCR analysis of cytokine gene expression in rats. 1994b. J. Immunol. Meth. 177: 23-28). CTLA4-Ig, which is a fusion protein, consisting of CTLA-4 (cytotoxic T cell late antigen) and the Fc-region of the IgG-antibody, binds to the B7 molecules (surface marker on antigen presenting cell, important for activation of T cells) of the antigen-presenting cells (APC) thus blocking the costimulatory signals (Sayegh, M. H.; Akalin, E.; Hanckock, W. W.; Russel, M. E.; Carpenter, C. B.; Turka, L. A.; CD28/B7 blockade after alloantigenic challenge in-vivo inhibits Th1 cytokines but spares Th2 . 1995. J. Exp. Med. 178: 1801-06). On completion of this short treatment (approx. 2 weeks) many models can boast a stable tolerance (Cobbold, S. & Waldmann, H.; How do monoclonal antibodies induce tolerance? A role for Infectious Tolerance? Annual Reviews Immunology 1998,16:619-644). Mediators of this tolerance are, presumably, regulatory CD4-cells. It could be shown that transfer of these cells to syngeneic animals could also induce tolerance. This phenomenon was first described in 1993 as “infectious tolerance” (Qin, S.; Cobbold., S. P.; Pope, H.; Elliott, J.; Kioussis, D.; Davies, J.; Waldmann, H.; Infectious transplantation tolerance. Science, 1993, 259: 974-977). But these experiments were only successful in a weak “rejection model”. With the help of a non-depleting mab against CD4 (RIB 5/2 monoclonal antibody directed against CD4 molecule), the same effect could be shown in a strong rejection model (Onodera, K.; Lehmann, M.; Akalin, E.; Volk, H. D.; Sayegh, M. H.; Kupiec-Weglinski, J. W.; Induction of infectious tolerance to MHC-incompatible cardiac allografts in CD4 monoclonal antibody-treated sensitized rat recipients. J. Immunol. 1996a, 157: 1944-1950). Using semiquantitative PCR a massively increased IL-4 (Interleukin-4) mRNA (messenger-ribonucleic acid) level in the transplanted organs (even after several adoptive transfers) could be shown. This is an indication for the importance of Th2 cytokines, especially Interleukin-4.

Apart from IL-4 several other cytokines are able to modulate Th1-mediated immune reactions. IL-4 induces the differentiation of naive CD4⁺ T cells in Th2 cells, is being produced by them and inhibits strongly the secretion of IFN-γ. Thus the generation of a Th1 immune response is suppressed (Banchereau, J.; Defrance, T.; Galizzi, J. P.; Miossec, P.; Rousset, F.; Human interleukin 4. Bull. Cancer 1991; 78: 299-306). IL-10, which is mainly produced by monocytes/macrophages and T cells, has a number of anti-inflammatory properties. It was shown, among other things, that:

i) IL-10 inhibits the expression of the MHC (major histocompatibility complex) class II on monocytes;

ii) it inhibits the production of inflammatory cytokines such as IFN-γ, IL-1 and IL-8 and TNF-α (tumor necrosis factor -α) and

iii) suppresses the proliferation of alloactivated lymphocytes (De Waal Malefyt, R.; Abrams, J.; Bennett, B.; Figdor, C. G.; De Fries, J. E.; Interleukin-10 (IL-10) inhibits cytokine synthesisi by human monocytes: an autoregulatory role of IL-10 produced by monocytes. J. Exp. Med. 1991. 174: 1209-1220; De Waal Malefyt, R., Haanen, J.; Spits, H.; Roncarolo, M. G.; Te Felde, A.; Figdor, C.; Johnson, C.; Kastelein, R.; Yssel, H.; De Fries, J. E.; Interleukin-10 (IL-10) and viral IL-10 strongly reduce antigen-specific human T cell proliferation by diminishing the antigen-presenting capacity of monocytes via downregulation of class II major histocompatibility complex expression. J. Exp. Med. 1991, 174: 915-924; 1991; Ralph, P.; Nakoinz, I.; Sampson, J. A.; Fong, S.; Lowe, D.; Min, H. Y.; Lin, L.; IL-10, T lymphocyte inhibitor human blood cell production of IL-1 and tumor necrosis factor. J. Immunol. 1992. 148: 808-814; Cassatella, M. A; Meda, L.; Bonora, S.; Ceska, M.; Constantin, G; Interleukin-10 (IL-10) inhibits the release of proinflammatory cytokines from human polymorphonuclear leucocytes. Evidence for an autocrine role of tumor necrosis factor and IL-1 beta in mediating the production of IL-8 triggered by lipopolysaccharide. J. Exp. Med. 1993. 178: 2207-2211; Qin, L., Ding, Y.; Pahud, D. R.; Robson, N. D.; Shaked, A.; and Bromberg, J. S.; Adenovirus-mediated gene transfer of viral interleukin-10 inhibits the immune response to both alloantigen and adenoviral antigen. Hum. Gene. Ther. 1997 8: 1365-1374).

There are also descriptions of an additional T-cell independent differentiation factor for Thh1 cells: IL-12. This cytokine is being secreted by macrophages and B-cells and leads to an increase in the IFN-γ production of NK (natural killer), CD4⁺ T cells and CD8⁺ T cells and thus induces the differentiation of naive CD4⁺ T cells into Th1-cells (Hsieh, C. S.; Macatonia, S. E.; Tripp, C. S.; Wolf, S. F.; O'Garra, A.; Murphy, K. M.; Listeria-induced Th1 development in aβ-TCR transgenic CD4⁺ T cells occurs through macrophage production of IL-12. Science 1993. 260: 547-551; Germann, T.; Gately, M. K.; Schoenhaut, D. S.; Lohoff, M.; Mattner, F.; Fischer, S.; Jin, S. C.; Schmitt, E.; Rude, E.; Interleukin-12/Tcell stimulating factor, a cytokine with multiple effects on T helper type1 (Th1) but not on Th2 cells. Eur. J. Immunol. 1993. 23: 1762-1768; Kennedy, M. K.; Picha, K. S.; Shanebeck, K. D,; Anderson, D. M.; Grabstein, K. H.; Interleukin-12 regulates the proliferation of Th1, but not Th2 or Th0, clones. Eur. J. Immunol. 1994, 24: 2271-2278). IL-12 is a heterodimeric glycoprotein, consisting of a 40 kDa (p40) and a 35 kDa (p35) subunit. “kDa” stands for kilo-Dalton. The IL-12p40 subunit is able to specifically inhibit the effects of the heterodimer. According to Mattem et al. (Mattner, F.; Fischer, S.; Guckes, S.; Jin, S.; Kaulen, H., Schmitt, E.; Rude, E.; Germann, T.; The interleukin-12 subunit p40 specifically inhibits effects of interleukin-12 heterodimer Eur. J. Immunol. 1993. 23: 2202-2207) supernatants of COS cells, transfected with murine IL12p40 gene, inhibit several IL-12 effects in-vitro. IL-12p40 inhibits the proliferation of PHA (phytohaemagglutinin) and IL-12 activated splenocytes.

In experimental and clinical investigations with allotransplants it could be shown that, in the phase of acute rejection, various cytokines are being expressed, whose cellular origin can be traced to Th1-cells, Th2-cells, cytotoxic CD8⁺ T cells, but also to non-lymphocytic cells (macrophages, endothelial cells, mast cells) (Dallman, M. J.; Larsen, C. P.; Morris, P. J.; Cytokine gene transcription in vascularised organ grafts: analysis using semiquantitative polymerase chain reaction. J. Exp. Med. 1991 174: 493-496). There are indications, that Th1 cytokines play a key role in the pathogenesis of acute transplant rejection. These are based on investigations of the cytokine expression pattern in grafts of tolerant animals. Indications for this can be found in experiments concerning the effective tolerance induction with anti-CD4 monoclonal antibodies (Benjamin, R. J.; Qin, S. X.; Wise, M. P.; Cobbold, S. P.; Waldmann, H.; Mechanisms of monoclonal antibody-facilitated tolerance induction: a possible role for the CD4 (L3T4) and CD11a (LFA-1) molecules in self-non-self discrimination. Eur. J. Immunol. 1988 18: 1079-1088; Takeuchi, T.; Lowry, R. P.; Konieczny, B.; Heart allografts in murine systems: the differential activation of Th2-like effector cells in peripheral tolerance. Transplantation. 1992. 53: 1281-1294; Siegling, A.; Lehmann, M.; Riedel, H.; Platzer, C.; Brock, J.; Emmrich, F.; Volk, H.-D.; A nondepleting anti-rat monoclonal antibody which suppresses T helper 1-like but not T helper 2-like intragraft lymphokine secretion induces long-term survival of renal allografts. Transplantation 1994a, 57: 464-467). The mechanism is not yet entirely understood, especially as a depletion of CD4⁺ T cells is not necessary for tolerance induction. A tolerance induction through anti-CD4 treatment is associated with a marked suppression of Th1 cytokine expression in the graft. This leads to the conclusion that Th1 cytokines play an essential role in the rejection of allografts (Siegling, A.; Lehmann, M.; Riedel, H.; Platzer, C.; Brock, J.; Emmrich, F.; Volk, H.-D.; A nondepleting anti-rat monoclonal antibody which suppresses T helper 1-like but not T helper 2-like intragraft lymphokine secretion induces long-term survival of renal allografts. Transplantation 1994a, 57: 464467; Lehmann M.; Graser E.; Risch K.; Hancock W. W.; Muller A.; Kuttler B.; Hahn H. J.; Kupiec-Weglinski J. W.; Brock J.; Volk H.-D.; Anti-CD4 monoclonal antibody-induced allograft tolerance in rats despite persistence of donor-reactive T cells. Transplantation Oct. 27, 1997; 64(8):1181-7).

The significance of Th2 cytokines is not as clear. In tolerant animals the Th2 response alone did not lead to a rejection of the graft. The persistence of Th2 cytokines in the grafts of the animals could be seen as an epiphenomenon, but it could also be the decisive factor for an inhibition of the Th1 response, which would make it a decisive criterion for the transplant situation. This assumption is confirmed by the observation that a temporary imbalance of Th1/Th2-cytokines immediately after the contact with the antigen can lead to a permanent modelling of the immune response (Scott, P.; T cell subsets and T cell antigens in protective immunity against experimental eishmaniasis. Curr. Top. Microbiol. Immunol. 1989. 155: 35-39). First experiments in-vitro show in the transplantation model, that the function of graft-infiltrating cells can also be influenced. The frequency of cells producing IFN-γ could be lowered by 50-70% under the influence of recombinant IL-4 (Merville, P.; Pouteil-Noble, C.; Wijdenes, J.; Potaux, L.; Touraine, J. L.; Banchereau, J.; Detection of single cells secreting IFN-gamma, IL-6 and IL-10 in irreversible rejected human kidney allografts and their modulation by IL-2 and IL-4. Transplantation 1993. 55: 639-642). From this we can deduce the hypothesis that a temporary overexpression of IL-4 at the site of the alloresponse, can result in acceptance of the graft.

The overexpression of IL-4 after ex-vivo gene-transfer, carried out with the help of recombinant adenoviruses, results in a significant prolongation of the graft acceptance in the allogeneic kidney transplant model of the rat in comparison to untreated grafts or to grafts treated with a reporter construct (Kato, H.; Ritter, T.; Ke, B.; Busuttil, R. W.; Kupiec-Weglinski, J. W; Gene transfer of IL-4 prolongs rat renal allograft survival and inhibits p²¹ras dependent activation pathway. Surg. Forum 1999a. 50: 381-383). Nevertheless, there are controverse opinions in the relevant literature, regarding the role of IL-4 in the induction of tolerance towards allogeneic grafts. It was shown, for example, that a local overexpression of IL-4, caused by adenovirally transduced or IL-4 transgenic grafts respectively, does not result in a prolongation of the graft acceptance (Smith, D. K.; Korbutt, G. S.; Suarez-Pinzon, W. L.; Kao, D.; Rajotte, R. V.; Elliott, J. F.; Interleukin-4 or interleukin-10 expressed from adenovirus-transduced syngeneic islet grafts fails to prevent beta cell destruction in diabetic NOD mice. Transplantation 1997.64 :1040-1049; Mueller, R.; Davies, J. D.; Krahl, T.; Sarvetnick, N.; IL-4 expression by grafts from transgenic mice fails to prevent allograft rejection. J. Immunol. 1997. 159:1599-603). On the other hand it has been shown that transgenic IL-4 producing grafts or the systemic application of IL-4 in combination with cyclosporin A treatment can result in a prolonged survival of allogeneic grafts (Takeuchi T.; Ueki, T.; Sunaga S.; Ikuta K.; Sasaki Y.; Li B.; Moriyama N.; Miyazaki J.; Kawabe K.; Murine interleukin 4 transgenic heart allograft survival prolonged with down-regulation of the Th1 cytokine mRNA in grafts. Transplantation 1997; Jul 15;64(1):152-7; Rabinovitch, A.; Suarez-Pinzon, W. L.; Sorensen, O.; Rajotte, R. V.; Power, R. F.; Combination therapy with cyclosporine and interleukin-4 or interleukin-10 prolongs survival of synergeneic pancreatic islet grafts in nonobese diabetic mice: islet graft survival does not correlate with mRNA levels of type 1 or type 2 cytokines, or transforming growth factor-beta in the islet grafts. Transplantation 1997. 64:1525-1531). However, quite often there is no satisfactory description of the methods used, which makes it impossible to decide whether methodical problems influenced the results.

In contrast to IL-4, the significance of IL-10 for the prolongation of graft acceptance is less disputed. Thus it could be shown that the overexpression of TGF-β1 (transforming growth factor) and vIL-10 (viral interleukin-10), an Epstein-Barr-Virus (EBV) encoded homologue to human or murine IL-10, results in a prolongation of graft acceptance in different allogeneic heart transplant models (Qin, L.; Chavin, K.; Ding, Y.; Favaro, J. P.; Woodward, J. E.; Lin, J.; Tahara, H.; Robbins, P.; Shaked, A.; Ho, D. Y.; Sapolsky, R. M.; Lotze, M. T.; Bromberg, J. S.; Multiple vectors effectively achieve gene transfer in a murine cardiac transplantation model. Transplantation 1995, 59: 809-816; Josien et al., 1998). Kato et al. could show, that the co-application of IL-4 and vIL-10 using recombinant adenoviruses, lead to a significant prolongation of the survival of allogeneic kidney grafts in a strong rejection model (Kato, H.; Fieblinger, C.; Ke, B.; Li,J.; Volk, H. D.; Busuttil, R. W.; Kupiec-Weglinski, J. W.; Ritter, T.; Synergistic effects of cytokine gene transfer in high responder rat renal allograft recipients. Transplantation 1999b 67: S 570 (Abstr. 112). It is interesting to note that the application of IL-4 alone had no influence on the prolongation of graft acceptance in this model.

Additionally, the overexpression of IL-12p40 seems to have a positive effect on the survival of grafts. Thus it could be shown that the local application of IL-12p40 inhibits the Th1-mediated immune response and prevents the rejection of allogeneic myoblasts, which were transfected with the cDNA (complementary desoxysibonucleic acid) for IL12p40 (Kato, K.; Shimozato, O.; Hoshi, K.; Wakimoto, H.; Hamada, H.; Yagita, H.; Okumura, K.; Local production of the p40 subunit of ineleukin 12 suppresses T-helper 1-mediated immune responses and prevents allogeneic myoblast rejection. Proc. Natl. Acad. Sci. 1996. 93: 9085-9089). Similar results were obtained in the model of islet cell transplantation in diabetic mice, where the overexpression of IL-12p40 prevented the Th1-mediated autoaggression (Rothe, H.; O'Hara Jr., R. M.; Martin, S.; Kolb, H.; Suppression of cyclophosphamide induced diabetes development and pancreatic Th1 reactivity in NOD mice treated with the interleukin (IL)-12 antagonist IL-12(p40)₂, Diabetologica 1997. 40: 641-649). The co-application of IL-4 and IL-12p40 using recombinant adenoviruses prolonged the survival of allogeneic kidney transplants in a strong rejection model significantly (Kato, H.; Fieblinger, C.; Ke, B.; Li, J.; Volk, H. D.; Busuttil, R. W.; Kupiec-Weglinski, J. W.; Ritter, T.; Synergistic effects of cytokine gene transfer in high responder rat renal allograft recipients. Transplantation 1999b 67: S 570 (Abstr. 112)).

The main problem of many experiments on the cytokine transfer still is the application of the cytokine. The systemic application of a cytokine can never create a local environment that corresponds to the physiological situation. Furthermore, cytokines in serum have a very short half-life, which means that the therapeutic protein would have to be provided constantly to achieve the desired serum level (H.-D. Volk, personal communication).

With adenovirus-mediated gene transfer of the donor organ, the cytokine expression in the graft can be enhanced, though it is usually only brief and in no way dependent on activation.

In contrast, retrovirally transduced T cells are able to express a protein durable and long lasting (Blaese, R. M.; Culver, K. W.; Miller, D.; Carter, C.; Fleisher, T.; Clerici, M.; Shearer, G.; Chang, L.; Chiang, Y.; Tolsthev, P.; Grennblatt, J. J.; Rosenberg, S. A.; Klein, H.; Berger, M.; Mullen, C. A.; Ramsey, W. J.; Muul, L.; Morgan, R. A.; Anderson, W. F.; T-Lymphocyte-Directed Gene Therapie for ADA-SCID; Intial Trial Results After 4 Years. Science 1995, 270:475-477). Especially activated T cells show an increased expression of their transgene (Quinn, E. R.; Lum, L. G.; Trevor, K. T.; T cell activation modulates retrovirus-mediated gene expression. Human Gene Therapie 1998, 9:1457-1467; Hammer, M. H.; Flugel Seifert, M.; Lehmann, M., Brandt, C.; Volk, H. D.; Ritter, T.; Potential of allospecific gene-engineered T cells in transplantation gene therapy: specific T cell activation determines transgene expression in vitro and in vivo. Hum. Gene. Ther. 2000 11: 1303-1311).

Bromberg et al. describe in Transplantation, Vol. 59, 6, 809-816, 1995, a method for a retroviral and adenoviral gene transfer directly into the graft. Unfortunately, it is not possible to protect the patient from exposure to the recombinant viruses.

### SUMMARY OF THE INVENTION

It is an object of the present invention to provide for a novel method to prevent the allogeneic graft rejection that has none of the drawbacks of the known ways and methods.

This and other objects have been achieved by the present invention the first embodiment which includes an in-vitro gene-modified T cell, obtained by stimulating a T cell of a graft recipient in-vitro with a cell of a graft donor or with a cell that expresses a dominant MHC molecule, and simultaneously or later, transfecting with a therapeutic gene using gene transfer.

In another embodiment the present invention relates to a method of using the in-vitro gene-modified T cell, comprising:

applying in-vivo said in-vitro gene-modified T cell to an allogeneic graft, thereby preventing an allogeneic graft rejection.

### BRIEF DESCRIPTION OF DRAWINGS

FIG. 1 shows the inhibition of proliferation of naïve T cells.

FIG. 2 shows the inhibition of proliferation of naïve T cells.

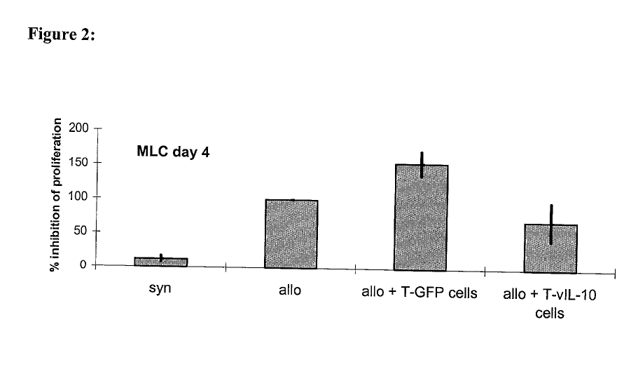

FIG. 3 shows the inhibition of interferon-γ production in naïve T-lymphocytes.

### DETAILED DESCRIPTION OF THE INVENTION

In detail the challenge to find new ways to prevent the allogeneic graft rejection was met by stimulating T cells of the graft recipient in-vitro with cells of the graft donor or with cells that express the dominant MHC molecules (major histocompatibility coupler). Simultaneously or later, T cells of the transplant recipient are also transduced with the help of gene transfer techniques with immuno-modulatory (therapeutic, e.g. viral IL-10, e.g. derived from EBV or CMV (cytomegalovirus), IL-4, IL12p40) genes. Following the gene transfer the transduced T cells start to express the immuno-modulatory genes. The gene transfer can be accomplished either with the help of retroviruses or with non-viral methods (liposomes, gene guns). The chosen experiment set-up leads to the generation and expansion of the allospecific transduced T cells in-vitro. After their in-vivo application during the transplantation of an allograft, the modified T cells have the property, due to their allospecificity, to migrate after the in-vivo application into the allogeneic graft as well as into the draining lymph nodes, where they then express the immuno-modulatory (therapeutic) genes.

The present invention makes it possible to prevent the rejection of allogeneic grafts (cells, tissues, organs) successfully and is therefore an effective way to induce tolerance and to maintain the tolerance towards allogeneic grafts (cells, tissues, organs) in transplantation medicine.

This means that the cells, modified according to the present invention, migrate into the graft due to their allospecificity. It turned out that, by producing IL-4, IL-10 and IL-12p40, a local environment of Th2 cytokines or Th1 antagonists, respectively, is created directly at the site of the antigen contact, depending on the level of activation of the invented cells. This means that we succeeded to generate IL-4, IL-10 or IL-12p40 producing, alloreactive T cells in-vitro using retroviral gene transfer.

The focus is on the expression of the transgene in the transplanted tissue itself. So far such cells (generated T cells) have been used mainly for the fight against tumours. For this the ex vivo generated tumour-specific T cells were transfected with proinflammatory cytokines (such as TNF-alpha), which “fought” the tumour and its metastases when they infiltrated it.

Grafts stressed by ischemia/reperfusion, infection or rejection episodes, also express autologous stress proteins, which can result in an immune response (e.g. heat shock protein HSP 70, specific autoreactive T cells). These cells can also be generated in-vitro and can be used as biological “drug delivery” system. As an alternative to the directly alloreactive T cells (against donor MHC molecules) indirectly alloreactive T cells (against donor peptides which are being presented by the recipient MHC) can be generated and used. Both approaches have the advantage of a lower reactivity against the graft in comparison to the directly alloreactive T cells.

According to the invention, the in-vitro transduced, gene-modified T cells are produced by co-culture or co-incubation with cell culture supernatants of so-called amphotrophic cell lines, which produce, for example, the recombinant retroviruses with the therapeutic transgenes. The procedure for the production of the vitro transduced, gene-modified T cells, according to our invention consists of the following steps:

generation of the packaging cell lines, which produce the recombinant retroviruses capble of gene transfer, which encode for therapeutic transgenes (through transduction).

generation of the alloreactive T cells in-vitro.

This means the cell line, which produces the retrovirus capable of gene transfer with the therapeutic transgene is being cultured. Additionally the lymphocytes (donor T cells or cells, which express the dominant MHC molecule and recipient T cells) are isolated from the whole blood. The donor T cells or the cell lines, which express the dominant MHC molecules, have to be irradiated to prevent a proliferation of these cells. After that a co-culture consisting of the mixed lymphocyte culture (primary MLC, mixed lymphocyte culture) and of the retrovirus-producing packaging cell line is carried out. The retroviral gene transfer can also be carried out by only using the virus supernatant of the packaging cell line, which is added to the culture of the lymphocytes (donor T cells or cells, which express dominant MHC molecules and donor T cells), so that a co-cultivation with the packaging cell line is not necessary.

In the case of the use of the non-viral gene transfer methods the co-culture of the mixed lymphocyte culture (donor T cells or cells, which express dominant MHC molecules and recipient T cells) with the packaging cell line is not necessary. With the help of non-viral gene transfer methods the plasmids, encoding for the therapeutic gene, the originating or originated alloreactive T cells are directly transduced in-vitro.

The alloreactive T cells can be used as “universal” vehicles for therapeutic genes. These consist preferably of the following:

Gene products, which are secreted by the cell and then exercise their immunoregulatory influence on other cells, e.g. alloreactive or graft-infiltrating cells, preferably cytokines (IL-13, cytokines which are homologous to the IL-10 gene, e.g. the CMV IL-10, cytomegalovirus IL-10);

Gene products, which are expressed on the cell surface of the regulatory T cells and which develop their immuno-regulatory effect through interaction with other cells, (alloreactive or graft-infiltrating cells), such as CTLA-4 (cytotoxic T cell late antigen) or genes which belong to the family of the notch-ligands/receptors, e.g. hSerrate (homo sapiens serrate), hDelta1 (homo sapiens delta (drosophila)-like 1) and Notch1-4 (homo sapiens notch (drosophila) homologous 1-4);

Gene products which are expressed intracellulary and which give the regulatory T cells a longer life span because of their cell-protective effect (e.g. anti-apoptotic genes such as bcl-2 (B cell leukemia-2), bcl-xl (bcl-2 homologue), bag-1 (bcl-2 associated athanogene which interacts with bcl-2);

Cell-protective genes (e.g. anti-apoptotic genes, heat shock genes).

IL-4, IL-10, vIL-10 and IL-2 p40 are preferred as therapeutic genes (transgenes). Hemoxygenase-1 is particularly preferred.

The transduced, gene-modified T cells can be used in various applications (iv, ip), different combinations thereof and/or different dosages at various time points.

The in-vitro transduced, gene-modified T cells, according to the invention, are suitable for the prevention of allogeneic graft rejection in-vivo and are preferably employed for the transplantation of allogeneic cells, tissue and organs. The in-vitro transduced, gene-modified T cells of the present invention are preferably used for: the transplantation of stem cells, bone marrow, skin, kidney, heart, liver, lung, cells of the central nervous system or islets of Langerhans. This means that T cells of the graft recipient are being stimulated in-vitro by cells of the graft donor or by cells, which express dominant MHC-molecules, while they are being transduced using gene transfer, i.e. immuno-modulatory genes are transferred. The result is the generation of T cells, which can induce or maintain tolerance towards allogeneic grafts.

The essence of the invention is the combination of amphotrophic cell lines, retroviral vectors, mixed lymphocyte cultures with a co-culture, consisting of mixed lymphocyte culture (primary MLC) and the cell line, which produced the therapeutic retrovirus. This leads to the genesis of gene-modified T cells, which can express therapeutic genes and which can also migrate, due to their allospecificity, into the allograft as well as into the draining lymph nodes. This method is successful, because it prevents the rejection of allogeneic grafts (cells, tissues, organs) effectively, thus providing an effective method for transplantation medicine.

According to the invention the main use for the therapeutic T cells is the prevention of allogeneic graft rejection. The in-vitro transduced, gene-modified T cells can be used as a means to induce and to maintain tolerance towards allogeneic grafts (cells, tissues, organs) and for the stimulation of the T cells of the graft recipient.

Having generally described this invention, a further understanding can be obtained by reference to certain specific examples which are provided herein for purposes of illustration only, and are not intended to be limiting unless otherwise specified.

### EXAMPLE

### EXAMPLE 1

### Production of the Therapeutic T-cell Lines

### Generation of the Cell Lines

At first, the cell lines which express the recombinant retroviruses with the therapeutic transgenes have to be produced. As a starting point for the production of infectious, replication-deficient retrovirus the cell-line PT 67 is used, which is derived from NIH/3T3 (murine fibroblasts, Retropack™, Clontech). PT 67 contain the genes gag, pol und env (10A1-stem) of the Moloney Murine Leukemia Virus (MoMuLV). The cells are grown in Dulbeccos' modified Eagle's Medium (DMEM), 10% foetal calf serum (FCS), 4 mM L-glutamin, 100 U/ml penicillin and 100 μg/ml streptomycin at 37° C. and in a 5% CO₂-atmosphere. The transfection of these cell lines with a retroviral vector, which does not contain the abovementioned genes, but does contain a therapeutic gene and a packaging signal, makes it possible to produce a replication-deficient retrovirus (i.e. the virus can infect its target cell, but can't replicate and infect other cells) (Mulligan, R. C.; The basic science of gene therapy. Science. 1993.260: 926-932; Ausubel, L. J; Kwan, C. K.; Sette, A.; Kuchroo, V.; Hafler, D. A.; Complementary mutations in an antigenic peptide allow for crossreactivity of autoreactive T-cell clones. Proc. Natl. Acad. Sci. U S A. 1993. 93: 15317-15322). The transfection of the PT67-cells is done, via calcium phosphate-transfection following standard protocols (Sambrook, M.; Fritsch, E. F.; Maniatis, T.; Molecular cloning: a laboratory manual; CSH Laboratory press, 2^{nd} edition, 1989).

In one embodiment a standard protocol having the following procedure has been followed: 1×10⁶ PT67-cells were seeded in 6 cm dishes one day prior to transfection in 5 ml medium containing 10% FCS. 24 hours later (cells should be 60-70% confluent) medium was changed 2-4 h prior to transfection by replacing the medium with 4.5 ml fresh medium. Then calcium-phosphate precipitate was prepared by adding 10 μg plasmid DNA encoding for the therapeutic gene to 31 μl 2 M CaCl₂ in a volume of 250 μl distilled water. Subsequently, 250 μl of 2×HBSP-buffer (50 mM HEPES, 10 mM KCl, 12 mM Dextrose, 280 mM NaCl, 1,5 mM Na₂HPO₄) was added. After 20 min of incubation at room temperature the precipitate was added directly dropwise to the 4,5 ml medium and cells were further incubated at 37° C. and 5% CO₂. After 12-16 hours the medium was replaced and G418 selection (0.5 mg/ml) was started by replacing the cell culture supernatant with fresh medium containing G-418.

Through selection with G 418 (0,5 mg/ml) clones and deriving cell lines are established, which produce the replication-deficient retrovirus as well as the therapeutic gene. In the case of IL-4 and IL-10 ELISA (enzyme linked immunosorbent assay) tests can be used to find the cell lines with the highest production of the therapeutic gene, these cell lines are then used in all further experiments. In the case of IL-12p40 there is no ELISA test available. Here the biological activity is determined in the Bioassay (Inhibition of the IFN-γ production with activated spleen cells).

### Example 2

### Generation of the Alloreactive T Cells In-vitro

One to two days before starting the mixed lymphocyte culture (irradiated donor T cells cultivated with T cells of the recipient), the cell line, which produces the recombinant retrovirus with the therapeutic transgene, is taken in culture (DMEM+10%FCS+selection antibiotic G 418 0,5 mg/ml final concentration).

On day 1, the co-culture consisting of the mixed lymphocyte culture (primary MLC) and the retrovirus-producing cell lines is started. To this end the cells of the cell line are trypsinized, centrifuged for 5 min at 1.200 rpm and then added to T cell medium (TCM) without FCS. After that the cells are counted and put into 96 well plates (2×10⁵-2×10⁶ cells/per well). Then the cells are left to grow in the CO₂-incubator for 3-4 h (5% CO₂) at 37° C., before the T cells are added.

The T cells of the graft recipient were previously isolated from peripheral blood with the help of a ficoll density separation according to standard protocols.

In one embodiment a standard protocol having the following procedure has been followed: 3 ml of pre-warmed (37° C.) ficoll solution were added to a red cap centrifuge tube. Then, isolated spleen cells in a culture medium were carefully put on top of the ficoll solution (while avoiding to mix ficoll and spleen cell solution). After centrifugation (400 g, 40 min, 20° C.), the upper layer was removed using a clean Pasteur pipette, leaving the lymphocyte layer undisturbed at the interface between medium and ficoll. Subsequently, the lymphocyte layer was transferred to a new sterile centrifuge tube using a new clean Pasteur pipette. In order to obtain enough cells it is important to harvest everything from the interface but it is also important to avoid contamination of the harvested cells with minimal amounts of both ficoll and culture supernatant. Then lymphocytes were washed three times and resuspended in the medium appropriate to the desired application.

For the antigen-presentation the cells of the graft donor are also isolated according to standard protocols. If cell lines are used, which express dominant MHC epitopes these are defrosted 1-2 days earlier and then cultivated in a CO₂-incubator until further use.

The cells, which are used for antigen-presentation, (as stimulator cells for the T cells of the graft recipient) have to be irradiated for 10 minutes with 30 gy (gray) before they are added to the mixed lymphocyte culture, to inhibit undesired proliferation.

After the irradiation of the antigen-presenting cells, they are centrifuged and resuspended in 20 ml TCM without FCS. After that the cells are counted and 50 μl TCM with 3.5×10⁵-4×10⁵ cells each of the graft recipient and the antigen-presenting cells, after addition of 3% autologous serum and 4 μg/ml polybren, are put into 96 well round bottom plates (total volume 100 μl) and incubated at 37° C. in the CO₂-incubator without any disturbance.

Day 4:

On day four, the MLC are transferred from the round bottom plates to flat bottom plates. First approximately 50 μl of the culture supernatant are taken away with a pipette (to be discarded) then the T cells are transferred with 2 to 3 resuspensions without air bubbles into a 96 well flat bottom plate. Additionally 100 medium (+hrIL-2, 25 U/ml), which should be prepared freshly, are added. The cells are then cultivated for a further 48 h at 37° C. under CO₂-atmosphere (5%).

Day 6:

On day 6, the G 418 selection is carried out, that means that all cells, which were not transduced during the retroviral gene transfer will perish during the G 418 selection. This means that only those cells that were transduced by the retrovirus survive the G 418 selection. From this stage onwards the cells have to be cultivated using G 418 (0,4 mg/ml G-418 final concentration). The cells are then cultivated in this medium for a further 48 h.

Day 8 Restimulation 2″

On day eight, after the first stimulation, the restimulation of the cells takes place. To this end either PBMC (peripheral blood mononuclear cells) of the graft donor or cell lines, which express the dominant MHC epitope, are used, as described for the first stimulation. The cells are irradiated again (10 min, 30 Gy), then centrifuged (1.200 rpm, 5 min) and then added to 20 ml TCM without FCS and the cells are counted. For the restimulation 100 μl are taken from the 96 well microtiter plate, which contains the MLC-cells, then 6×10⁶ stimulator cells are added. Additionally an environment of 3% autologous serum and a G 418 concentration of 0.4 mg/ml is established. Then the cells are incubated for two more days.

Day 10:

On day 10, fresh TCM medium is added (+hrIL-2, 25 U/ml) (take away 100 μl and then add 100 μl TCM medium (+hrIL-2, 25 U/ml)+G 418, 0.4 mg/ml). Then the cells are incubated for two more days.

Day 12:

The cell culture plates should now contain proliferating cells (blasts), which can be multiplied using further restimulation steps, to harvest a sufficient number of cells for the application in the clinic. On day 14, there is also the possibility to purify and to isolate the generated blasts using a special ficoll density separation (Ficoll 3000). 24 h after the gradient the next restimulation (3″) takes place.

For the stimulation either PBMC-cells of the graft donor or cell lines, which express dominant MHC molecules or cell lines, which are transfected with genes for these molecules and express these constitutively (K. Wood, personal communication) can be used.

Example 3: Alternatives to co-culture

Instead of the co-culture with amphotrophic cell lines, only cell culture supernatants of the amphotrophic cell line containing retroviruses are used for the transduction of the T cells.

### Example 4: The Bioassay

The existence of the therapeutic gene in the supernatant can be proved by:

IL-4, ELISA, MHC-II upregulation on spleen cells;

vIL-10, ELISA, inhibition of the TNF-α production through macrophages, reduction of the MHC-II expression on monocytes; and

IL-12p40, no ELISA, inhibition of the production of IFN-γ after stimulation of spleen cells.

### Example 5

### Immuno-regulatory Potential of the Allospecific T_{VIL-10} Lymphocytes

The inhibition of the proliferation of naive T cells with the help of lymphocytes transgenic for vIL-10

was first proven in-vitro in the mixed lymphocyte culture (MLC). This invitro system was meant to imitate the situation of the T cell reactivity after allogeneic organ transplantation. To achieve this, naive recipient cells (which are meant to represent the T cells of the graft recipient) were stained with the membrane dye SNARF™ on day 0. When a cell divided this dye was handed down evenly, so that the intensity of fluorescence decreased.

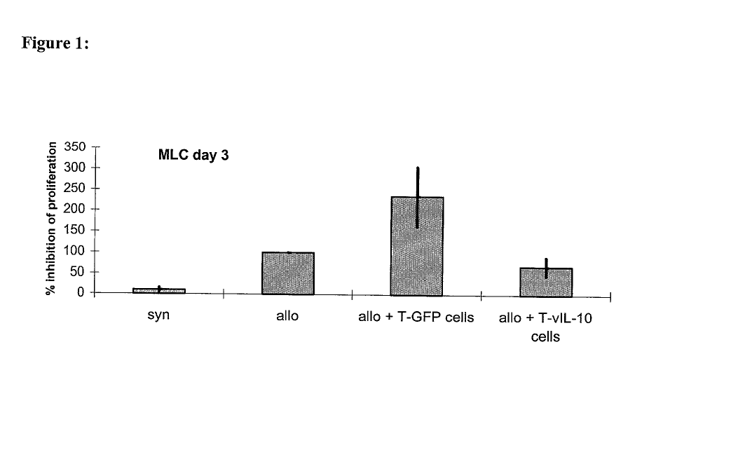

Following this the stained recipient cells and the irradiated stimulator cells (which are meant to imitate the graft-specific cells) were put into a 96 well flat bottom plate at a ratio of 1:1 (3,5×10⁵ cells each). To investigate the influence of the therapeutic TVIL-10 T cells on the antigen-induced proliferation of the naive lymphocytes, these were added at a ratio of 1:20 (5%) to the experiment. As control, a syngeneic control was used and, additionally allospecific T_{EGFP} lymphocytes (with an irrelevant control gene, Enhanced Green Fluorescent Protein, as so-called therapeutic gene), were used at the same ratio. The intensity and decline of fluorescence intensity was measured using flow cytometry (FACS) on days 1-4. In comparison to the allogeneic control without therapeutic T cells, an inhibition of the proliferation to approx. 70-80% shown in the experiments with TVIL-10 lymphocytes on day 3 and 4 (FIGS. 1 und 2).

### Example 6: Inhibition of the Interferon-γ Production in Naïve T-lymphocytes with the Help of T_{VIL-10} Cells

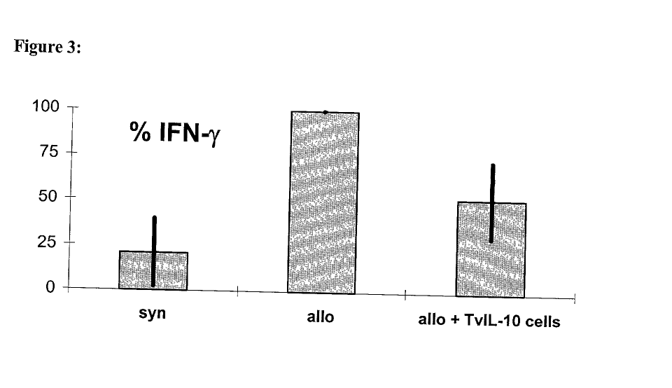

The inhibition of the interferon-γ production in naive T-lymphocytes with the help of T_{VIL-10} cells was also proven in the MLC. The same approach was used. In this case another membrane dye (CFSE), which lights up in the fluorescence channel 1 of the flow cytometer, was used. The detection of IFN-γ was done using a PE-marked monoclonal intracellular antibody against IFN-γ in the FACS on day 4. The decline of the IFN-γ production in naïve T-lymphocytes after cultivation is 50% on day 4 (FIG. 3).

A comparison of the transgenic T_{VIL-10} cells with T_{EGFP} cells and non-transgenic allospecific lymphocytes on the protein and RNA level provides information on whether the therapeutic cells differ in their cytokine expression patterns (rIL-2, rIFN-γ, rIL-10 and so on) from other cells. Furthermore activation markers (rCD25), apoptotic (FasL) and antiapoptotic (bag-1) gene expression patterns are analysed. These experiments help to characterise the therapeutic cells distinctly and to analyse possible modes of action of these lymphocytes.

### Example 7

### Gene Transfer into Alloreactive T Cells Using Non-viral Methods

Apart from the viral gene transfer, the non-viral gene-transfer for the generation of alloreactive, gene-modified T cells is examined. To this end, the allospecific T cells produced in the mixed lymphocyte culture are incubated, with e.g. certain liposome formulations, which contain the plasmid with the therapeutic gene, or treated with a gene gun. Preferred examples for the liposome formulations are cationic lipids, such as quartary ammonium detergents, cationic derivatives of cholesterol and diacylglycerol, lipid derivatives of polyamines, high molecular weight polymers and dendrimers.

The co-culture with a virus-producing packaging cell line is not necessary for this approach.

### Example 8

### Gene Transfer into Alloreactive T Cells with Other Viral Vector Systems

Apart from the retroviral gene transfer based on murine Moloney Leukemia Virus (MoMuLV), alloreactive, gene-modified T cells can be produced with the help of other viral vector systems. This includes the gene transfer with lentiviral constructs (this means retroviruses based on the Human Immunodeficiency Virus (HIV), constructs on the basis of adeno-associated viruses (AAV) and constructs on the basis of cytomegaloviruses (CMV)).

German patent application 100 28 833.2, filed Jun. 9, 2000; PCT/DE01/02184, filed Jun. 8, 2001; and the patents and literature references cited in the specification are incorporated herein by reference.

Obviously, numerous modifications and variations on the present invention are possible in light of the above teachings. It is therefore to be understood that within the scope of the appended claims, the invention may be practiced otherwise than as specifically described herein.

1. An in-vitro gene-modified T cell, obtained by stimulating a T cell of a graft recipient in-vitro with a cell of a graft donor or with a cell that expresses a dominant MHC molecule, and simultaneously or later, transfecting with a therapeutic gene using gene transfer.

2. The in-vitro gene-modified T cell according to claim 1, wherein said T cell is an alloreactive T cell.

3. The in-vitro gene-modified T cell according to claim 1 produced by:
a) culturing a cell line which produces a retrovirus that is suitable for gene transfer and expresses a therapeutic gene;
b) isolating a lymphocyte from whole blood, the spleen or a lymph node;
wherein said lymphocyte is an irradiated donor T cell, an irradiated cell which expresses the dominant MHC molecule or a recipient T cell; and
c) either co-culturing a mixed lymphocyte culture and the cell line, or exclusively culturing a supernatant containing retrovirus which is used for said transfecting.

4. The in-vitro gene-modified T cell according to claim 3, wherein said retrovirus is a moloney murine leukemia virus or a lentivirus.

5. The in-vitro gene-modified T cell according to claim 1, produced by isolating a lymphocyte from whole blood, the spleen, or a lymph node;
wherein said lymphocyte is an irradiated donor T cell, an irradiated cell which expresses the dominant MHC molecule, or a recipient T cell;
wherein an allospecific T-cell produced using a mixed lymphocyte culture is incubated with a liposome formulation containing the plasmid with the therapeutic gene or treated with a gene gun.

6. The in-vitro gene-modified T cell according to claim 1, wherein said therapeutic gene is a cytokine, an interleukin, a notch-ligand/receptor, or a cell-protective gene.

7. The in-vitro transfected T cell according to claim 6, wherein said therapeutic gene is IL-4, IL-10, viral IL-10, IL-12p40, IL-13, hemoxygenase-1, CTLA-4, hSerrate-1, hDelta-1, notch 1-4, bcl-2, bcl-xl, or bag-1.

8. A process for the generation of a gene-modified T cell, comprising: stimulating a T cell of a graft recipient in-vitro with a cell of a graft donor or with a cell which expresses a dominant MHC molecule; and
concurrently or later transfecting an immuno-modulatory therapeutic gene via gene-transfer.

9. The process according to claim 8, wherein the T cell is an alloreactive T cell.

10. The process according to claim 8, wherein:
a) culturing a cell line which produces a retrovirus that is suitable for gene transfer and expresses a therapeutic gene;
b) isolating a lymphocyte from whole blood, the spleen or a lymph node;
wherein said lymphocyte is an irradiated donor T cell, an irradiated cell which expresses the dominant MHC molecule or a recipient T cell; and
c) either co-culturing a mixed lymphocyte culture and the cell line, or exclusively culturing a supernatant containing retrovirus which is used for said transfecting.

11. The process according to claim 10, wherein the retrovirus is a moloney murine leukemia virus or a lentivirus.

12. The process according to claim 8, wherein lymphocytes are isolated from whole blood, the spleen, or a lymph node;
wherein said lymphocyte is an irradiated donor T cell, an irradiated cell which ex-presses the dominant MHC molecule, or a recipient T cell;
wherein an allospecific T-cell produced using a mixed lymphocyte culture is incubated with a liposome formulation containing the plasmid with the therapeutic gene or treated with a gene gun.

13. The process according to claim 8, wherein said therapeutic gene is a cytokine, an interleukin, a notch-ligand/receptor, or a cell-protective gene.

14. The process according to claim 13, wherein said therapeutic gene is IL-4, IL-10, viral IL-10, IL-12p40, IL-13, hemoxygenase-1, CTLA-4, hSerrate-1, hDelta-1, notch 1-4, bcl-2, bcl-xl, or bag-1.

15. A method of using the in-vitro gene-modified T cell according to claim 1, comprising:
applying in-vivo said in-vitro gene-modified T cell to an allogeneic graft, thereby preventing an allogeneic graft rejection.

16. The method according to claim 15, wherein a tolerance towards said allogeneic graft is induced and/or maintained.

17. The method according to claim 15, wherein a T cell of a graft recipient is stimulated.
